(19) United States
(12) Patent Application Publication
(10) Pub. No.: US 2002/0170078 A1
(43) Pub. Date: Nov. 14, 2002

(54) Calcineurin-related transgenic mammals, compositions and methods

(76) Inventors: Isabelle Mansuy, Mulhouse (FR); Eric R. Kandel, Riverdale, NY (US)

Correspondence Address: Cooper & Dunham, LLP, 1185 Avenue of the Americas, New York, NY, 10036, US

(21) Appl. No.: 10/091,714

(22) Filed: Mar. 5, 2002

Related U.S. Application Data
(60) Provisional application No. 60/274,290, filed on Mar. 8, 2001.

(51) Int. Cl. A01K 67/027; C12N 15/85; C12N 5/06

(52) U.S. Cl. 800/8; 800/14; 435/455; 435/325; 435/320.1

(57) ABSTRACT

The present invention provides a transgenic nonhuman mammal and mammalian cells whose genomes encode a calcineurin inhibitor. The expression of the inhibitor is targeted to the forebrain and may be induced experimentally. The present invention further provides a composition of matter comprising the nucleic acid sequences encoding the inhibitor and additional sequences required for its targeted expression and regulation. In addition, the present invention provides methods for determining whether an agent inhibits long-term potentiation and/or learning and memory in a mammal.

This application claims priority of U.S. provisional application Serial No. 60/274,290, filed Mar. 8, 2001, the content of which is hereby incorporated into this application by reference.

The invention disclosed herein was made with U.S. government support under grant number HL-54591 from the National Institutes of Health, Heart Lung and Blood Institute.

Throughout this application, various publications are referenced by author and date. Full citations for these publications may be found at the end of the specification immediately preceding the claims. The disclosures of these references in their entireties are hereby incorporated by reference into this application to describe more fully the art to which this invention pertains.

### BACKGROUND OF THE INVENTION

The threshold for hippocampal-dependent synaptic plasticity and memory storage is thought to be determined by the balance between protein phosphorylation and dephosphorylation mediated by the kinase PKA and the phosphatase calcineurin.

Studies of the molecular mechanisms of learning and memory in vertebrates and invertebrates have illustrated the existence of both positive and negative regulators of synaptic plasticity and memory storage (Abel et al., 1998). For example, long-term forms of plasticity and memory in invertebrates can either be enhanced or suppressed by changing the ratio between activator and represser isoforms of the transcription factor cAMP response element binding (CREB) protein (Bartsch et al., 1995; Yin et al., 1995). Another potential site for the control of synaptic plasticity and memory, which operates upstream of the transcriptional machinery, resides in the balance between the phosphorylation and dephosphorylation of specific substrates (Soderling and Derkach, 2000).

On the one hand, kinases such as the Ca2+/calmodul-independent protein kinase II (CaMKII), the cAMP-dependent protein kinase A (PKA), and the mitogen-activated protein kinase (MAPK) are positive regulators, critical for the initiation of many forms of synaptic plasticity and for learning and memory (Lisman, 1994; Kornhauser and Greenberg, 1997; Abel et al., 1998; Lee et al., 2000). On the other hand, phosphatases such as the Ca2+/calmodulin-dependent calcineurin (CN) and protein phosphatase 1 (PP1) are thought to be negative regulators inhibiting both synaptic plasticity and memory storage (Ikegami et al., 1996; Mansuy et al., 1998a; Winder et al., 1998; Ikegami and Inokuchi, 2000).

Initial clues to the antagonistic role of kinases and phosphatases came from pharmacological and genetic studies that indicated that PKA is required for the expression of persistent LTP because of its role in suppressing an endogenous phosphatase cascade (Blitzer et al., 1995; Abel et al., 1997). Consistent with this idea, overexpression of constitutively active calcineurin in the forebrain of transgenic mice was found to impair an intermediate and PKA-dependent phase of LTP, as well as the transition from short to long-term memory and memory retrieval (Mansuy et al., 1998a, 1998b; Winder et al., 1998). These results suggested that calcineurin can act as an inhibitory constraint on PKA-dependent processes and down regulate pathways supporting synaptic plasticity and memory.

### SUMMARY OF THE INVENTION

The present invention provides a transgenic nonhuman mammal whose germ or somatic cells contain (i) a first heterologous nucleic acid sequence encoding a transcriptional activator whose expression is under the control of a CaMKIIα promoter and (ii) a second heterologous nucleic acid sequence encoding a protein whose expression is under the control of a promoter responsive to the transcriptional activator in a regulatable manner.

The present invention also provides a mammalian cell comprising (i) a heterologous first nucleic acid sequence encoding a transcriptional activator whose expression is under the control of a CaMKIIα promoter and (ii) a second heterologous nucleic acid sequence encoding a protein whose expression is under the control of a promoter responsive to the transcriptional activator in a regulatable manner.

The present invention further provides a composition of matter comprising (i) a first nucleic acid encoding a transcriptional activator whose expression is under the control of a CaMKIIα promoter and (ii) a second nucleic acid encoding a protein whose expression is under the control of a promoter responsive to the transcriptional activator in a regulatable manner.

The present invention also provides methods for determining whether an agent inhibits long-term potentiation in a mammal comprising (i) administering the agent to the transgenic mammal of the present invention, wherein the first heterologous nucleic acid encodes rtTA and the second heterologous nucleic acid encodes an inhibitor of calcineurin whose expression is regulated by a tetracycline-responsive sequence, and wherein doxycycline has been administered to the mammal; (ii) measuring the resulting long-term potentiation in the mammal; and (iii) comparing the long-term potentiation so measured to the long-term potentiation measured in a control transgenic mammal to which doxycycline, but no agent, has been administered, a decrease in long-term potentiation relative to the control mammal indicating that the agent inhibits long-term potentiation in a mammal.

The present invention also provides a method for determining whether an agent inhibits long-term potentiation in a cell comprising (i) contacting a hippocampal sample from the brain of the transgenic mammal of the present invention with the agent, wherein the first heterologous nucleic acid encodes rtTA and the second heterologous nucleic acid encodes an inhibitor of calcineurin whose expression is regulated by a tetracycline-responsive sequence, and wherein the cells of the sample have been exposed to doxycycline; (ii) measuring the resulting long-term potentiation in the hippocampal sample; and (iii) comparing the long-term potentiation so measured to the long-term potentiation measured in a hippocampal sample from a control transgenic mammal, wherein the cells of the sample have been exposed to doxycycline, but no agent, a decrease in long-term potentiation relative to the control mammal indicating that the agent inhibits long-term potentiation in a mammal.

The present invention further provides a method for determining whether an agent inhibits long-term memory formation, retention or recall in a mammal, which comprises (i) administering the agent to the transgenic mammal of the present invention, wherein the first heterologous nucleic acid encodes rtTA and the second heterologous nucleic acid encodes an inhibitor of calcineurin whose expression is regulated by a tetracycline-responsive sequence, and wherein doxycycline has been administered to the mammal; (ii) measuring the memory formation, retention, or recall of the mammal via a behavioral test; and (iii) comparing the memory formation, retention, or recall so measured to that of a control transgenic mammal to which doxycycline, but no agent, has been administered, a decrease in memory formation, retention, or recall relative to the control mammal indicating that the agent inhibits memory formation, retention, or recall.

### BRIEF DESCRIPTION OF THE FIGURES

FIG. 1A: Strategy to obtain doxycycline-dependent expression of the CN inhibitor in brain. Transgenic mice carrying the rtTA gene driven by the CaMKIIα promoter were crossed with mice carrying the tetO promoter linked to the CN autoinhibitory (Al) gene. Double transgenic (mutant) mice from this crossing express the CN inhibitor when fed doxycycline. Without doxycycline, rtTA does not activate gene transcription. pA: polyadenylation signal sequence.

FIG. 1B: RT-PCR showing transgene expression (263 bp band) in extracts from hippocampus and cortex from adult mutants on doxycycline (M on). No expression was detected in controls treated or not treated with doxycycline (C), in untreated mutants (M), or in mutants treated with doxycycline then withdrawn from the drug (M on/off). GAPDH indicates equivalent DNA concentrations in the reactions.

FIG. 1C: Reduced CN activity in extracts from hippocampus (Hip) and cortex from adult mutants on doxycycline (Hip: n=7 versus control (n=3) or mutant (n=5), p<0.05, asterisk; cortex: n=6 versus control (n−2) or mutant (n=3), p<0.05, asterisk). CN activity returned to basal levels in mutants on/off doxycycline (Hip: n=3 versus mutant on doxycycline or control, p<0.05; cortex: n=3 versus mutant on doxycycline or control, p<0.05).

FIG. 1D: In situ hybridization on a sagittal brain section from an adult mutant on doxycycline showing mRNA in the Hip, dentate gyrus (Dg), cortex (Cx), olfactory bulb (Ob), striatum (St), and cerebellum (Ce). No expression was detected in the absence of doxycycline (mutant).

FIG. 2A: LTP elicited by a single, 1 s 100-Hz train in mutant and control slices (12 slices, 7 mice in each group) Inset: top traces are responses during tetanization in controls (left) and mutants (right). For clarity of presentation, stimulus artifacts were removed. Lower traces are baseline fEPSPs just before tetanus superimposed with responses 40 min after tetanus.

FIG. 2B: 1-train LTP with KT5720 (1 uM) in mutants on doxycycline (7 slices, 4 mice) or untreated mutants (5 slices, 4 mice), and in control on doxycycline (4 slices, 3 mice) or untreated controls (15 slices, 8 mice).

FIG. 2C: Similar to (A) except performed with DL-AP5 (100 uM, at least 20 min pretreatment, control, 10 slices, 3 mice; mutant, 9 slices, 3 mice).

FIG. 2D: Input-output curve of synaptic transmission in slices from mutants and controls on doxycycline (16 slices, 6 mice each).

FIG. 2E: Saturation of LTP with four 1 s 100 Hz trains with 5 min interval delivered twice 20 min apart in slices from controls (n=6).

FIG. 2F: LTP elicited by four, 1 s 100-Hz trains with 5 min interval in controls (9 slices, 7 mice) and mutants (13 slices, 7 mice).

FIG. 3A: Responses to 3 min of 5-Hz stimulation (control, 5 slices, 2 mice; mutant, 6 slices, 2 mice).

FIG. 3B: LTD elicited by 15 min of 1-Hz stimulation. Overall responses in 13 slices from 5 controls and 5 mutants. Insets: scatterplots of responses 30 min after stimulation.

FIG. 3C: LTD elicited by 15 min of 1-Hz stimulation. Responses in 10 slices from 5 controls and 5 mutants, excluding 3 slices in each case that gave responses over 100% baseline. Insets: scatterplots of responses 30 min after stimulation.

FIG. 3D: Reversal of 1-train LTP enhancement in mutants on/off doxycycline (22 slices, 11 mice). Control on/off, 22 slices, 11 mice.

FIG. 3E: PPF at various interstimulus intervals is increased in slices from mutants on doxycycline versus controls on doxycycline (10 slices, 3 mice in each case). *p<0.05, **p<0.01, ***p<0.001, one way ANOVA (p<0.0001).

FIG. 3F: Reversal of PPF increase in slices from mutants on/off doxycycline (6 slices, 3 mice). Control on/off, 6 slices, 3 mice.

FIG. 4A: CA1 LTP in anesthetized mice. Test stimuli were delivered at 0.033 Hz at an intensity that evoked 50% maximal fEPSP slope. 50 stimuli at 100 Hz (arrow) induced a larger transient increase in fEPSP slope in mutant (n=4) than in control (n=5) mice on doxycycline. 1 hr after the initial tetanus, 2 trains of 50 stimuli at 100 Hz (arrows) induced enhanced LTP in mutant (n=4) versus control (n=3) mice on doxycycline. Insets: representative field potentials 10 min after the first (left) or the second (right) tetanus. Potentiated responses (black) are superimposed on control responses (gray).

FIG. 4B: Dentate gyrus LTP in anesthetized mice. Test stimuli were delivered at 0.033 Hz at an intensity that evoked a population spike amplitude of 1-3 mV. Tetanic stimulation (arrow, 6 series of 6 trains of 6 stimuli at 400 Hz, 200 ms between trains, 20 s between series) induced larger LTP in mutant (n=3) versus control (n=3) mice on doxycycline.

FIG. 4C: Dentate gyrus LTP in awake mice. Test stimuli and tetanic stimulation as in FIG. 4B, with 20 min recording sessions apart from the first hour after LTP induction. More persistent LTP in mutant (n=4) than in control (n=4) mice on doxycycline 2 and 3 days after induction. Asterisks indicate significant difference in mean level of potentiation over the time period indicated by brackets, p<0.05 by Student's t test.

FIG. 5A: Reversible improvement of short and long-term memory on the object recognition task. Mean exploration during training. Control (pooled), n=12; mutant, n=16; mutant on dox, n=16.

FIG. 5B: Reaction to the spatial change. Increased re-exploration of the displaced object (plain, left three bars) in mutants on dox (n=16, versus control (pooled), n=11 or mutant, n=16, all p<0.05, asterisk). No re-exploration of the undisplaced objects (hatched, right three bars) was observed. Re-exploration was calculated by subtracting the exploration time before and after the spatial change.

FIG. 5C: Reaction to a novel object. Discrimination ratio is the time spent exploring the novel object over total exploration time (for 3 objects, total time is the mean time for familiar plus time for novel). Ratio of 0.5 reflects equal exploration of all objects. Higher discrimination ratio indicating increased exploration of the novel object in mutants on dox (n=16, versus control (pooled), n=10, p<0.05, asterisk, or mutant, n=14, p<0.01).

FIG. 5D: Increased discrimination ratio in mutant mice on dox 5 min and 3 hr after introduction of the novel object with the complex protocol (mutant on dox, n=8 versus control (pooled), n=8, p<0.05, asterisk). No difference in exploration was observed between groups at 24 hr with the complex protocol (control (pooled), n=6; mutant on dox, n=6).

FIG. 5E: Increased discrimination ratio in mutant mice on dox 3 days after introduction of the novel object with the complex protocol (mutant on dox, n=10 versus control (pooled), n=9, p<0.02, asterisk) and 1 week (mutant on dox, n=10 versus control (pooled), n=14, p<0.05, asterisk) after introduction of the novel object with the simple protocol. No difference in exploration was observed between groups both at 24 hr and 2 weeks (control (pooled), n=8; mutant on dox, n=8) with the simple protocol.

FIG. 5F: Reversibility of the improvement. No difference after 5 min in re-exploration of the displaced object (plain, left two bars) or the undisplaced objects (hatched, right two bars).

FIG. 5G: Discrimination between the novel and the familiar objects in mutant and control mice on/off dox (control, n=7; mutant, n=11).

FIG. 6A: Mutants on doxycycline (n=12) have significantly lower escape latency across days on the Morris water maze (asterisk) from day 5 to day 10 during acquisition (mutant on doxycycline versus control (pooled), n=60, p<0.05 or mutant (n=16), p<0.01).

FIG. 6B: Mutants on doxycycline have significantly lower CSE (asterisk) from day 5 to day 10 during acquisition (mutant on doxycycline versus control, p<0.04 or mutant, p<0.01).

FIG. 6C: Mutants on doxycycline have significantly lower escape latency (asterisk) on day 12 during transfer (mutant on doxycycline versus control, p<0.04 or mutant, p<0.03).

FIG. 6D: Mutants on doxycycline have significantly lower CSE (asterisk) on day 12 during transfer (mutant on doxycycline versus control, p<0.03 or mutant, p<0.03).

FIG. 6E: Normal learning on a cued version of the Morris water maze.

FIG. 7A: Probe trials in the Morris water maze on day 5 of the acquisition phase. Mean number of platform crossings in the training quadrant (Position 1 during acquisition, 3 during transfer) and in corresponding zones in other quadrants. Significant increase in platform crossings (asterisk) in the first training quadrant in mutant on doxycycline (versus control, p<0.01 or mutant, p<0.05).

FIG. 7B: Probe trials in the Morris water maze on day 10 of the acquisition phase.

FIG. 7C: Probe trials in the Morris water maze on day 15 of the acquisition phase. Significant increase in platform crossings (asterisk) in the first training quadrant in mutant on doxycycline (versus control, p=0.01 or mutant, p=0.0001).

FIG. 7D: Mean errors and rank of the first error per day on the radial arm maze. No difference between mutant (n=8) or control (n=10) whether on doxycycline (on) or withdrawn from doxycycline after treatment (on/off).

### DETAILED DESCRIPTION OF THE INVENTION

The present invention provides a transgenic nonhuman mammal in which the expression of a calcineurin inhibitor is spatially restricted to the forebrain and temporally regulated by the addition of doxycycline. As demonstrated herein, the transient expression of a calcineurin inhibitor in a mouse using the instant methods results in enhanced learning and memory as measured in behavioral tests, as well as increased long-term potentiation, as measured in hippocampal neurons. Thus, the transgenic mammals, compositions, and methods of the instant invention may be used to specifically evaluate the potential adverse effects of particular agents on learning and memory. As such, the instant invention provides an important addition to current methods for the assessment neurotoxicity.

Definitions

As used herein, “CaMKIIα promoter” means all or an operative portion of the 8.5 kilobase genomic DNA fragment upstream of the mouse calcium-calmodulin-dependent protein kinase IIα, deposited with the American Type Culture Collection under ATCC Accession No. 98582.

As used herein, “heterologous”, in reference to a nucleic acid in an organism, means that the nucleic acid originated from a foreign species, or if from the same species, is substantially modified from its native form in composition and/or genomic locus by deliberate human intervention.

As used herein, “long-term potentiation”, also referred to herein as “LTP”, means an activity-dependent increase in synaptic strength. LTP can be measured both in vivo and in vitro by electrophysical techniques well-known in the art. LTP is known to be correlative with long-term memory in mammals.

As used herein, “long-term memory” refers to a type of memory distinct from short-term memory as understood in the art. For example, “long-term memory” is stored for an extended period of time compared to short-term memory, and is characterized by distinct molecular changes that do not occur in short-term memory. These molecular changes include protein synthesis, as well as synaptic remodeling, i.e., changes in the number and pattern of synaptic connections.

The terms “nucleic acid”, “polynucleotide” and “nucleic acid sequence” are used interchangeably herein, and each means a polymer of deoxyribonucleotides and/or ribonucleotides. The deoxyribonucleotides and ribonucleotides can be naturally occurring or synthetic analogues.

The terms “polypeptide,” “peptide” and “protein” are used interchangeably herein, and each means a polymer of amino acid residues. The amino acid residues can be naturally occurring or chemical analogues thereof. Polypeptides, peptides and proteins can also include modifications such as glycosylation, lipid attachment, sulfation, hydroxylation, and ADP-ribosylation.

As used herein, “promoter” means a region of DNA proximal to a transcription start site of a sequence and required for transcription of that sequence to occur. Promoters contain regulatory elements having binding sites for the core transcription machinery as well as for other proteins required to initiate transcription. A promoter may further contain sequences comprising binding sites for transcriptional activators. The activators may be tissue-specific or they may regulate transcription in more than one tissue. Thus, the presence of certain sequences in the promoter of a heterologous gene in a transgenic animal can confer a tissue-specific pattern of expression thereof in the animal.

As used herein, “regulatable”, in reference to the response of a promoter to a transcriptional activator, means that the response of the promoter to the transcriptional activator can be induced or suppressed by experimentally controlled means. For example, transcription of a gene under the control of the tetO promoter, in the presence of the tTA transcriptional activator, will be suppressed by the presence of doxycycline and, conversely, expression will occur in the absence of doxycycline.

As used herein, “tetO” means a cognate tetracycline operator sequence which can be present in a promoter. Thus, a “tetO promoter” means a promoter having one or more such sequences.

As used herein, “tTA” means a fusion protein comprising the repressor of the Tn10 tetracycline-resistance operon of Escheria coli and a C-terminal portion of protein 16 of herpes simplex virus that functions as a strong transcriptional activator. This fusion protein is a tetracycline-controlled transactivator. For example, tTA will bind to the tetO region of a promoter and function as a strong activator of transcription in the absence of doxycycline. Doxycycline may therefore be used to suppress transcription from a promoter having tetO sequences.

As used herein, “rtTA” means a protein that is a variant of the tTA protein, and plays a role opposite that of tTA, i.e., rtTA protein requires doxycycline in order to activate transcription from a tetO promoter. Doxycycline can thus be used as an activator of transcription in conjunction with rtTA and a transgene operatively linked to a teto-containing promoter.

As used herein, “vector” means any nucleic acid vector known in the art. Such vectors include, but are not limited to, plasmid vectors, cosmid vectors, and bacteriophage vectors.

Embodiments of the Invention

The present invention provides a transgenic nonhuman mammal whose germ or somatic cells contain (i) a first heterologous nucleic acid sequence encoding a transcriptional activator whose expression is under the control of a CaMKIIα promoter and (ii) a second heterologous nucleic acid sequence encoding a protein whose expression is under the control of a promoter responsive to the transcriptional activator in a regulatable manner.

In one embodiment of the instant invention, the transcriptional activator encoded by the first nucleic acid comprises rtTA and the second nucleic acid encodes a calcineurin inhibitor whose expression is regulated by a tetO promoter. Thus, the expression of the inhibitor may be induced in the forebrain of the transgenic mammal by administering doxycycline in the food for a period of time and in an amount sufficient to induce its expression.

In another embodiment of this invention, the transcriptional activator encoded by the first nucleic acid comprises tTA and the second nucleic acid encodes a calcineurin inhibitor whose expression is regulated by a tetO promoter. Thus, the expression of the inhibitor may be repressed in the forebrain of the transgenic mammal by administering doxycycline in the food for a period of time and in an amount sufficient to repress its expression.

The transgenic mammal of the instant invention may be, for example, a mouse, a rat, a sheep, a cow, a dog, a pig, or a primate.

The present invention also provides a mammalian cell comprising (i) a heterologous first nucleic acid sequence encoding a transcriptional activator whose expression is under the control of a CaMKIIα promoter and (ii) a second heterologous nucleic acid sequence encoding a protein whose expression is under the control of a promoter responsive to the transcriptional activator in a regulatable manner.

In one embodiment of this invention, the transcriptional activator encoded by the first nucleic acid comprises rtTA and the second nucleic acid encodes a calcineurin inhibitor whose expression is regulated by a tetO promoter. Thus, the expression of the inhibitor may be induced in the cell by administering doxycycline in the culture medium for a period of time and in an amount sufficient to induce its expression.

In another embodiment of this invention, the transcriptional activator encoded by the first nucleic acid comprises tTA and the second nucleic acid encodes a calcineurin inhibitor whose expression is regulated by a tetO promoter. Thus, the expression of the inhibitor may be repressed in the cell by administering doxycycline in the culture medium for a period of time and in an amount sufficient to repress its expression.

In a preferred embodiment, the cell is an oocyte, an embryonic stem cell or a neuronal cell.

An example of a calcineurin inhibitor is the carboxy-terminal autoinhibitory sequence of calcineurin, which is known in the art.

In order to induce or repress the expression of the nucleic acid that is regulated by the tetO promoter, one may, for example, administer doxycyline to a mouse at 6 mg/g food for at least one week. In tissue culture, one may, by way of an additional example, add doxycycline to the tissue culture medium at 6 ng/ml for at least 20 minutes.

The present invention also provides a composition of matter comprising (i) a first nucleic acid encoding a transcriptional activator whose expression is under the control of a CaMKIIα promoter and (ii) a second nucleic acid encoding a protein whose expression is under the control of a promoter responsive to the transcriptional activator in a regulatable manner. In a preferred embodiment, the two nucleic acids exist within a single nucleic acid molecule, such as a plasmid vector.

In an embodiment of this invention, the transcriptional activator encoded by the first nucleic acid of the composition of matter comprises rtTA or tTA. In a further embodiment, the second nucleic acid encodes a calcineurin inhibitor whose expression is regulated by a tetO promoter.

The present invention also provides a method for determining whether an agent inhibits long-term potentiation in a mammal, comprising (i) administering the agent to a transgenic mammal of the present invention, wherein the heterologous first nucleic acid encodes rtTA and the second heterologous nucleic acid encodes an inhibitor of calcineurin whose expression is regulated by a tetracycline-responsive sequence; (ii) measuring the resulting long-term potentiation in the mammal; and (iii) comparing the long-term potentiation so measured to the long-term potentiation measured in a control transgenic mammal to which doxycycline, but no agent, has been administered, a decrease in long-term potentiation relative to the control mammal indicating that the agent inhibits long-term potentiation in a mammal.

The present invention also provides a method for determining whether an agent inhibits long-term potentiation in a cell, comprising (i) contacting a cultured hippocampal sample with the agent, wherein the sample has been exposed to doxycycline and wherein the first heterologous nucleic acid encodes rtTA and the second heterologous nucleic acid encodes an inhibitor of calcineurin whose expression is regulated by a tetracycline-responsive sequence; (ii) measuring the resulting long-term potentiation in the sample; and (iii) comparing the long-term potentiation so measured to the long-term potentiation measured in a control sample to which doxycycline, but no agent, has been administered, a decrease in long-term potentiation relative to the control mammal indicating that the agent inhibits long-term potentiation in hippocampal neurons.

Long-term potentiation, or LTP, may be measured in either an awake or an anesthetized mammal by techniques well-known in the art. LTP may also be measured in vitro, using cultured hippocampal samples, by art-recognized techniques. Methods for culturing hippocampal samples for use in measuring LTP are well-known in the art.

The present invention also provides a method for determining whether an agent inhibits long-term memory formation, retention or recall in a mammal, which comprises (i) administering the agent to a transgenic mammal of the present invention, to which doxycycline has been administered, wherein the first heterologous nucleic acid encodes rtTA and the second heterologous nucleic acid encodes an inhibitor of calcineurin whose expression is regulated by a tetracycline-responsive sequence; (ii) measuring the memory formation, retention, or recall of the mammal via a behavioral test; and (iii) comparing the memory formation, retention, or recall so measured to that of a control transgenic mammal to which doxycycline, but no agent, has been administered, a decrease in memory formation, retention, or recall relative to the control mammal indicating that the agent inhibits memory formation, retention, or recall.

Behavioral tests for measuring long-term memory formation, retention or recall are well-known in the art. Examples of such tests which may be used in the instant methods include, without limitation, the Barnes circular maze, the novel object recognition task, an object exploration task, the Morris water maze and the 8-arm radial maze.

The agent used in the instant methods can be, for example, a polypeptide, a small molecule or a nucleic acid. Agents contemplated in this invention include, without limitation, industrial chemicals, food additives, pesticides, and fertilizers. Other examples may include elemental metals, such as lead, mercury, or arsenic.

This invention is illustrated in the Experimental Details section which follows. This section is set forth to aid in an understanding of the invention but is not intended to, and should not be construed to, limit in any way the invention as set forth in the claims which follow thereafter.

Experimental Details

Synopsis

A direct test of the idea that PKA and endogenous calcineurin serve to balance one another would require demonstrating that relief of the inhibitory constraint applied by calcineurin enhances both LTP and memory. To establish whether endogenous calcineurin acts as an inhibitory constraint in this balance, we examined the effect of genetically inhibiting calcineurin on plasticity and memory, using the doxycycline-dependent reverse tetracycline-controlled transactivator (rtTA) system to express a calcineurin inhibitor reversibly in the mouse brain. Transgenic mice were generated in which calcineurin activity is decreased by the expression of the inhibitor. To restrict the inhibitor expression both temporally and regionally in the brain, we used the system in combination with the promoter for CaMKIIα (Gossen et al., 1995; Mansuy et al., 1998b). With this system, we found that the regulated inhibition of calcineurin leads to a reversible facilitation of LTP both in vitro and in vivo in the Schaffer collateral pathway to the pyramidal cells of the CA1 region in hippocampus and in the perforant pathway to the dentate gyrus, and that the enhanced LTP is PKA-dependent. The facilitated LTP was accompanied by a reversible enhancement of several phases of spatial and nonspatial learning and memory. These experiments demonstrate that transient reduction of calcineurin activity in the adult brain is sufficient to enhance synaptic efficacy and memory storage reversibly, suggesting that calcineurin plays a determinant role in signaling pathways recruited in brain plasticity and memory. Further, our results provide direct evidence that calcineurin acts as a negative regulator of synaptic plasticity, and of learning and memory.

Methods

Generation of Transgenic Mice

A founder carrying the tetO-AI transgene was crossed to C57BI/6J then to CaMKIIα promoter-rtTA animals (Mansuy et al., 1998b). RT-PCR and in situ hybridization were performed as previously described using oligonucleotides specific for tetO-AI (Mansuy et al., 1998a). Phosphatase assays used a nonradioactive CN assay kit (Biomol. Research Laboratories). ANOVAs were used to compare activity between groups.

Treatment and Genotypes

For all experiments, doxycycline (Mutual Pharmaceutical Co., Philadelphia, Pa.) was administered at 6 mg/g food at least 1 week before experimentation. Control mice were either treated or not treated with doxycycline and for all behavioral tests, results from both groups were pooled. Control groups were wild type and mice carrying either one of the two transgenes.

Electrophysiology

The experimenter was blind to genotype and drug treatment throughout. Field potential recordings from area CA1 of mouse hippocampal slices were performed as previously described (Winder et al., 1996). Doxycycline was present in the ACSF at 6 ng/ml for recordings. For in vivo experiments, mice were anesthetized with urethane (1.8 mg/kg i.p. for nonrecovery) or sodium pentobarbitone (60 mg/kg i.p. during electrode implantation). For CA1 recordings, a bipolar stimulating electrode was positioned in one hippocampus and a micropipette recording electrode in the contralateral stratum radiatum. For dentate gyrus recordings, a stimulating electrode was placed in the medial perforant path and the recording electrode in the ipsilateral hilus. Nichrome wire electrodes were fixed with dental cement for recordings from awake mice (Errington et al., 1997). Test responses were evoked by 60 microseconds monophasic stimuli at 1 per 30 s. fEPSP potentiation is expressed as percentage change relative to the mean control response during the 10 min prior to tetanic stimulation. Population spike potentiation is expressed as millivolt change in spike amplitude. Data are given as mean±SEM.

Behavior and Data Analyses

For all behavioral tasks, mutant and control littermates (males, 3-5 months old) were used. Statistical analyses used ANOVAs with genotype as the between-subject factor, and session (exploration task), day, area (quadrant or platform in the Morris water maze), or treatment (8-arm radial maze) as within-subject factors. Mean±SEM are presented.

Object Exploration

Mice were habituated to the arena (63×51×25 cm) for 4 days then tested with either a complex or simple protocol. The complex protocol consisted of five successive 5-min training sessions with 5-min inter-trial interval (ITI) (Buhot and Naili, 1995) and a last 5-min session either 5 min, 3 hr, or 24 hr later. The simple protocol was of two 10-min sessions, 10-min ITI, and a last 5-min session either 24 hr, 3 days, 1 week, or 2 weeks later. The time spent exploring each object was recorded with a video tracking system (Viewpoint, France) tracing a phosphorescent tag on the animals' heads.

Morris Water Maze

The task was performed as previously described (Malleret et al., 1999) with three training phases: 2 days with a visible platform followed by 10 days (acquisition phase) with a hidden platform in the training quadrant, then 5 days (transfer phase) with the hidden platform in the opposite quadrant. For each phase, four trials, 120 s maximum and 15-min ITI were given daily, probe trials were 60 s. The animals' trajectories were recorded with a video tracking system (HVS Image Analyzing VP-118).

8-Arm Radial Maze

Food-deprived males (90% ad libitum weight) on doxycycline were habituated for a week to retrieve food pellets in cups placed at the end of each arm of an elevated 8-arm radial maze. Mice were tested for 1 week and allowed eight visits each day then doxycycline was removed and animals were tested again 2 weeks later. Errors (runs into an already visited arm) and rank of the first error were recorded.

Results

Doxycycline-Dependent Inhibition of Calcineurin

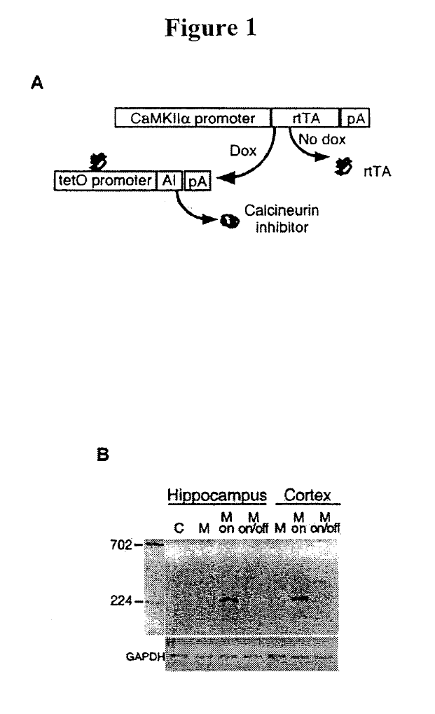

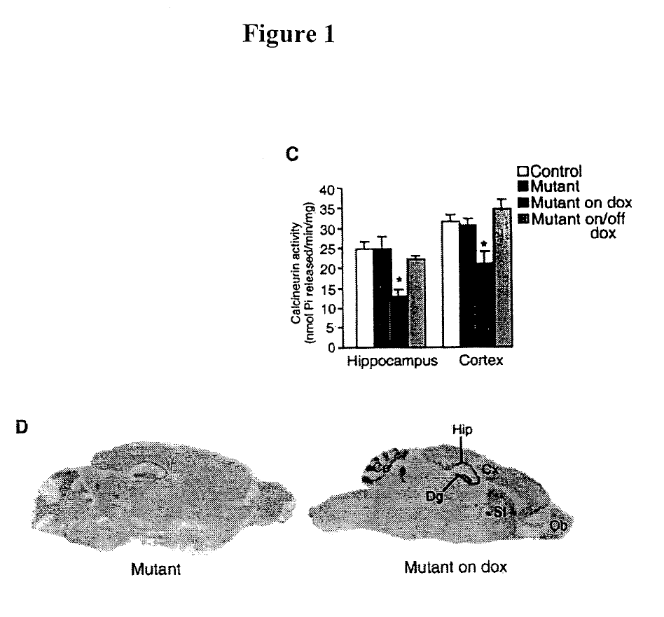

We achieved temporally restricted inhibition of CN in adult mouse brain by expressing the autoinhibitory domain of the C-terminus of calcineurin (Perrino et al., 1995) with the rtTA system Transgenic mice carrying the rtTA-responsive tetO promoter fused to the calcineurin inhibitor gene were crossed with mice expressing rtTA in neurons under the control of the CaMKIIα promoter (Mansuy et al., 1998b) (FIG. 1A). In double transgenic animals (mutants), doxycycline (dox) induced the expression of the calcineurin inhibitor.

Calcineurin inhibitor mRNA was detected in extracts from hippocampus and cerebral cortex from 3-week-old (data not shown) and adult (FIG. 1B) mutants on doxycycline while it was absent in tissue from control mice. In situ hybridization confirmed the presence of transgene mRNA in hippocampus and cortex, and revealed it also in striatum, olfactory bulb, and cerebellum (FIG. 1D). In both hippocampus and cortex, transgene expression resulted in a 35%-45% decrease in calcineurin activity (FIG. 1C, p<0.05). This decrease was reversed when transgene expression was suppressed by withdrawal of the mutant mice from doxycycline. Thus, 12 days after doxycycline removal following a 1-week treatment, transgene mRNA was no longer detected in hippocampus and cortex (FIG. 1B) and calcineurin activity returned to baseline (FIG. 1C). Finally, no gross anatomical or structural changes were observed after transient expression of the calcineurin inhibitor in adult brain (data not shown).

Inhibiting Calcineurin Facilitates LTP in vitro

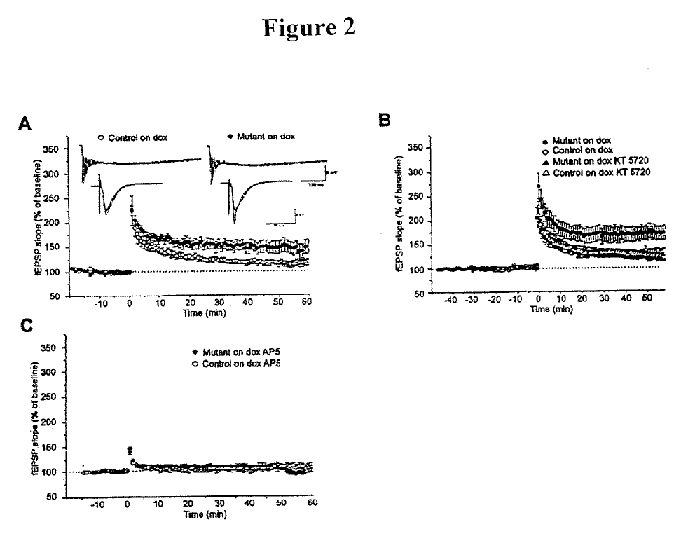

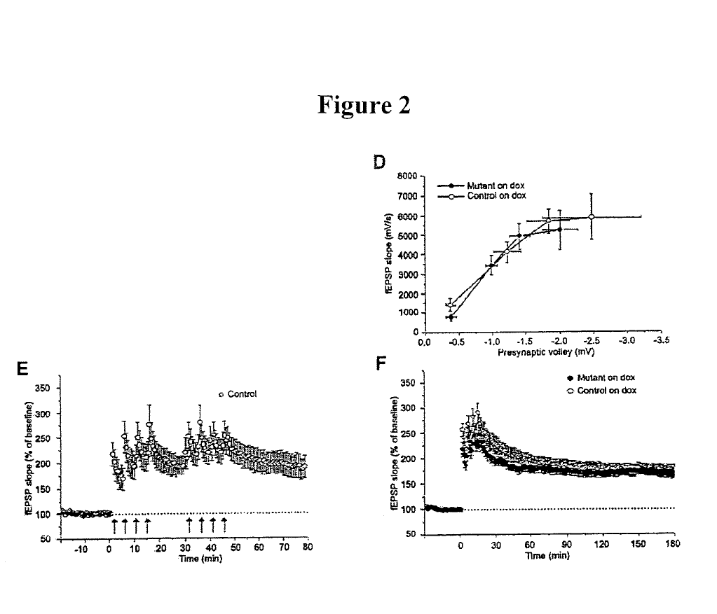

Calcineurin is thought to be critical for several forms of synaptic plasticity (Lisman, 1994; Mulkey et al., 1994; Winder et al., 1998; Zhuo et al., 1999; Lu et al., 2000). In particular, we have found that PKA-dependent forms of LTP are suppressed by calcineurin overexpression, suggesting that calcineurin and PKA act antagonistically to regulate a common component of LTP (Winder et al., 1998). If this were the case, one would predict that inhibiting calcineurin would facilitate LTP in a PKA-dependent manner. To test this idea, we examined LTP elicited with a single train of 100 Hz in the Schaffer collateral pathway to area CA1 of hippocampal slices. We observed that in slices from mutants expressing the calcineurin inhibitor, LTP was increased compared to controls (155±10 versus 121±6% base-line 30 min after tetanus, FIG. 2A, p<0.05). We next assessed whether this enhanced LTP was PKA dependent using the PKA inhibitor KT5720. Consistent with previous studies (Huang and Kandel, 1994; Abel et al., 1997), 30 min pretreatment with KT5720 had no effect on 1-train LTP in control slices (134±6% baseline 30 min after tetanus, FIG. 2B). However, in mutant slices, the increased potentiation was suppressed by KT5720 (168±17 versus 126±7% baseline 30 min after tetanus, FIG. 2B) suggesting it required PKA activity.

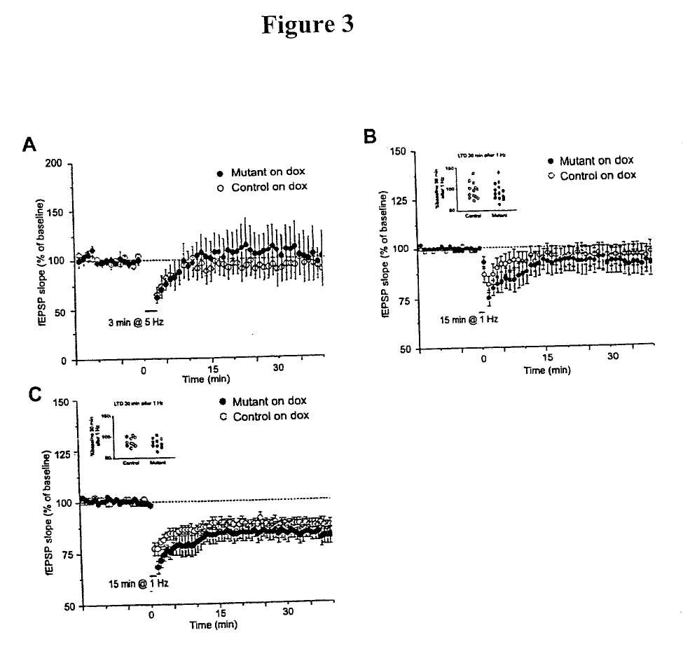

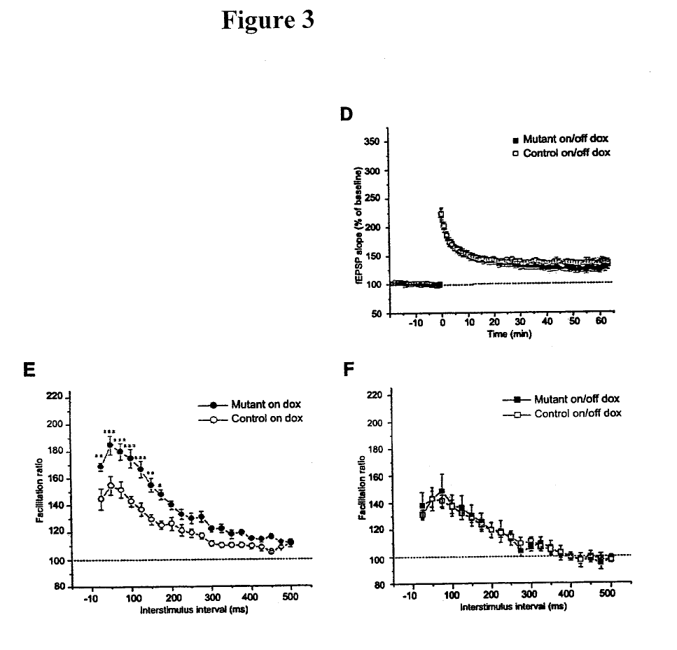

Four independent experiments suggested that the LTP enhancement did not represent a nonspecific alteration of synaptic transmission. In the first experiment, the enhanced LTP was dependent on N-methyl-D-aspartate receptor (NMDA-R), as it was abolished by the NMDA-R antagonist DL-AP5 (100 uM, FIG. 2C). Second, basal synaptic transmission was normal in mutants on doxycycline as assessed by stimulus-response curves of baseline fEPSPs versus presynaptic fiber volley sizes across a range of stimulus intensities (FIG. 2D) Third, the total depolarization elicited by the tetanus (estimated by the area under the fEPSPs during tetanus) was indistinguishable between mutant and control slices (inset FIG. 2A; control, 2876±391 mV; mutant, 2768±340 mV). Fourth, the LTP enhancement could not be explained by an increased basal probability of glutamate release resulting in greater depolarization during the tetanus since paired-pulse facilitation (PPF), an index of presynaptic activity and release probability, was markedly facilitated in mutants on doxycycline compared to controls, suggesting rather a reduced release probability in mutant mice (FIG. 3E).

The enhancement of LTP observed with one 100-Hz train may reflect a decrease in the threshold for the induction of LTP or an enhanced maintenance of LTP. To distinguish between these possibilities, we examined LTP induced with four 100-Hz trains. 4-train LTP was nondecremental and similar in slices from mutant and control mice on doxycycline (FIG. 2F). However, 4-train LTP was not further enhanced by another 4-train tetanization in slices from controls (FIG. 2E) or from mutants on doxycycline (data not shown), indicating that the evoked LTP was saturated. These data, therefore, are consistent with a role for calcineurin in regulating the induction of LTP.

Since pharmacological studies have suggested a role for phosphatases in synaptic changes elicited by low frequency stimulation (Mulkey et al., 1994; Thomas et al., 1996), we next determined the effect of the calcineurin inhibitor on responses induced by low frequency stimulation. As shown in FIG. 3A, three minutes of 5-Hz stimulation, a crossover frequency between those producing LTP and those producing long-term depression (LTD) (Bienenstock et al., 1982), resulted in responses that were not different from baseline in both control and mutant slices. Further, consistent with previous observations (Winder et al., 1998), 15 min of 1-Hz stimulation did not produce any response in slices from adult mice. By contrast, 15 min of 1-Hz stimulation induced very modest LTD in area CA1 that was similar in mutant and control slices from young animals (3.5-4.5 weeks, FIG. 3B). The modest averaged population LTD was in part due to the fact that this protocol elicited a lasting synaptic enhancement on occasion (inset FIG. 3B). The occasional enhancement of synaptic transmission that we observed (to a similar degree in both mutant and control populations) obscures the fact that in most individual experiments, LTD was observed. Indeed, when these individual experiments were excluded from re-analysis (three from both populations, inset FIG. 3C), robust LTD was observed, which again was indistinguishable between mutant and control slices (FIG. 3C). Thus, although we cannot rule out subtle differences in LTD induction between mutant and control slices, these data demonstrate that LTD can be induced in slices from mutant mice on doxycycline. These results corroborate recent genetic studies revealing normal LTD in slices from mice lacking CNAU (Zhuo et al., 1999), but they contrast with previous pharmacological studies indicating that LTD is blocked by calcineurin inhibition (Mulkey et al., 1994).

Finally, to determine whether the enhancement of 1-train LTP and PPF was a direct effect of the transgene, we assessed whether suppression of transgene expression in the adult could reverse this enhancement. Slices from mutants in which transgene expression was induced for 1 week then blocked by doxycycline removal for 2 weeks prior to experimentation (on/off doxycycline) displayed 1-train LTP (131±6% baseline 30 min after potentiation, FIG. 3D) and PPF (FIG. 3F) that was similar to control slices, indicating that the enhancement of LTP and PPF is a direct effect of the calcineurin inhibitor.

LTP is Also Enhanced in vivo in Both CA1 and Dentate Gyrus

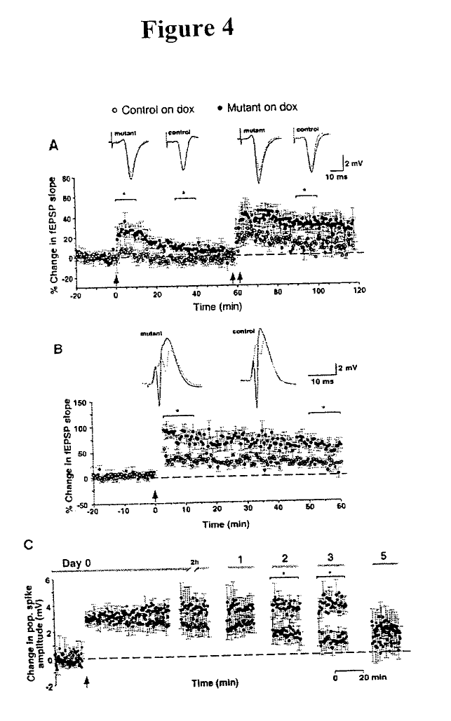

Previous studies of LTP in genetically modified mice have revealed discrepancies between results obtained in vitro or in vivo (Errington et al., 1997). We therefore examined LTP in vivo in both anesthetized and awake mice. In area CA1, weak tetanic stimulation (50 pulses at 100 Hz) of Schaffer collateral afferents induced a transient potentiation of the fEPSP slope in anesthetized mutants on doxycycline. In contrast, this tetanus produced only slight post-tetanic potentiation in control mice (FIG. 4A). A subsequent, stronger tetanus (two trains at 100 Hz) induced significant potentiation during the first 10 min after the tetanus in control mice (p<0.05, FIG. 4A). Consistent with the in vitro data, this initial potentiation was significantly greater in mutant versus control mice (p<0.05) so that 50-60 min after the tetanus, only mutant mice maintained significantly enhanced responses (p<0.05). Basal synaptic efficacy appeared normal in mutant mice since stimulus-response curves were indistinguishable from those in control mice (data not shown). These results are consistent with the facilitory effect on LTP observed in the rat after chronic injection of antisense oligodeoxynucleotides against calcineurin (Ikegami et al., 1996).

In contrast to the in vitro data, in vivo PPF was similar in mutant and control animals, with both groups showing maximal facilitation at an interstimulus interval of 30 ms (facilitation ratio: mutant, 183±9.9%; control, 191±14%, data not shown). At present, the cause of this difference between preparations is unclear. One possibility is that while in vivo recordings were performed in the dorsal 1/3 of the hippocampus, in vitro recordings were from slices of the middle 2/3 of the hippocampus. PPF was reported to be greater in more dorsal than ventral portions of the hippocampus (Papatheodoropoutos and Kostopoulos, 2000). Regardless, the fact that a similar modulation of LTP is seen in both preparations suggests that altered PPF does not play a critical role in the enhancement of LTP.

In the dentate gyrus of anesthetized mice, 400 Hz titanic stimulation induced a stable and significant LTP of fEPSP in all mice tested (FIG. 4B). As in CA1, LTP was significantly greater in mutant compared to control mice (p<0.05). Associated increases in population spike amplitude were also larger in mutants (by 4.9±1.5 mV, 50-60 min post-tetanus) than in controls (by 3.1±0.7 mV), although this enhancement of spike potentiation was not significant (data not shown). Stimulus-response curves and PPF again appeared normal in mutants (data not shown), once more indicating normal basal transmission and excitability.

We next examined the time course of LTP over several days in the dentate gyrus of awake animals with chronically implanted electrodes. LTP was induced using a pattern of tetanic stimulation identical to that in anesthetized mice. Population spike amplitudes were potentiated to similar extents one and three hours after LTP induction (FIG. 4C). However, spike amplitudes in mutant mice remained robustly elevated for at least 3 days after tetanic stimulation (p<0.05 versus control group) while in control mice, potentiation was no longer significantly different from baseline after 3 days. By 5 days, although spike amplitudes remained potentiated in mutant animals, there was no significant difference between genotypes. Potentiation is expressed as change in population spike amplitude since the initial fEPSP slope was often obscured by the spike in awake animals.

Calcineurin Inhibition Enhances Short-term Memory for Object and Spatial Configurations

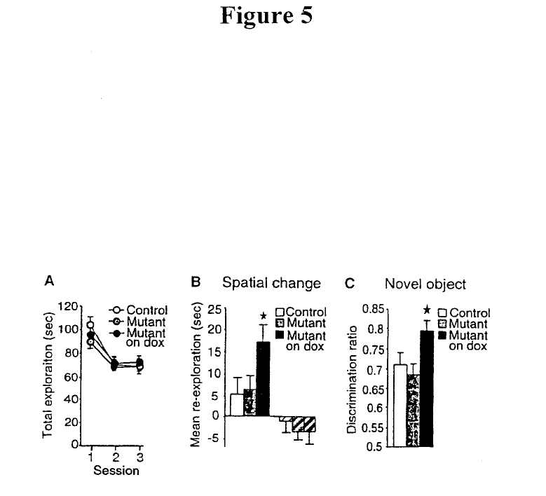

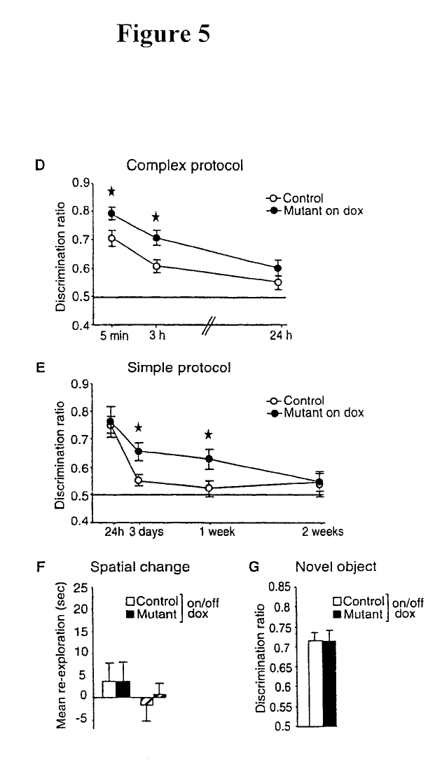

To examine whether a reduction in calcineurin activity leading to increased LTP might improve memory, we first tested the animals on a spontaneous object recognition task. This task is based on the discrimination between a familiar and a novel situation and requires the hippocampus (Vnek and Rothblat, 1996). We assessed the ability of the animals to discriminate either between a novel and a familiar spatial location of an object, or between a novel and a familiar object. Mice were first trained with a complex protocol for which they were placed in an arena containing three novel objects that they were allowed to explore for three sessions of 5 min each. In the first session, all groups spent a similar amount of time exploring the objects (25%-35% total time), then exploration decreased, indicating comparable habituation to the objects and attention in all groups (FIG. 5A). No difference in basic locomotor activity or in thigmotaxis, an index of anxiety characterized by motion along the walls, was observed between groups (data not shown).

Five minutes after these initial sessions of exploration, one object was moved to a novel location in the arena. In response to the change in objects configuration, all groups tended to re-explore the displaced object more than the nondisplaced objects, indicating they perceived the change in spatial arrangement (FIG. 5B). However, mutant mice on doxycycline spent significantly more time re-exploring the displaced object than did control mice [F(1,25)=4.272, p<0.05] or mutant mice off doxycycline [F(1,30) 4.495, p<0.05], suggesting enhanced memory for the initial configuration. No significant difference in re-exploration of the nondisplaced objects was observed between the groups.

Following a session of habituation to the novel configuration, one of the familiar objects was replaced with a novel object and a discrimination ratio was determined. After a 5 min delay, mutant mice on doxycycline showed increased discrimination between novel and familiar objects with a significantly stronger preference for the novel object compared to other groups [control, F(1,24)=5.287, p<0.05; mutant, F(1,28)=9.414, p<0.01]. However, all groups explored the novel object more than the familiar ones (FIG. 5C). These results suggest that memory for spatial location of object and for object is improved by the CN inhibitor at short retention intervals.

Intermediate to Long-term Memory is Also Improved by Calcineurin Inhibition

To assess whether enhanced memory could also be observed after longer retention intervals, the discrimination ratio was examined 3 hr and 24 hr after acquisition. Again, mutant mice on doxycycline displayed greater preference for the novel object than control mice after 3 hr [F(1,14}=8.18, p<0.05], although both groups explored the novel object preferentially (FIG. 5D). However, by 24 hr, the discrimination ratio was low in both groups (FIG. 5D). These results suggested that the calcineurin inhibitor improves memory not only at short but also at intermediate retention intervals.

Next, to examine whether long-term memory could also be improved, we trained the animals on a simple and more intense protocol. This protocol consisted of two training sessions with two objects, and after a retention interval of 24 hr, 3 days, 1 week, or 2 weeks, one familiar object was replaced with a novel object. We found that after 24 hr, both control mice and mutant mice on doxycycline displayed a large preference for the novel object, indicating robust memory for the familiar object (FIG. 5E). Strikingly after 3 days [F(1,17)=8.267, p<0.02] or after 1 week [F(1, 22)=4.375, p<0.05], mutant mice on doxycycline still clearly preferred the novel object while control mice did not show any preference for this object (FIG. 5E). Two weeks later, however, mutant mice explored both objects similarly, suggesting decayed memory for the familiar object (FIG. 5E). These results clearly indicated that the CN inhibitor allowed long-term memory to persist for over a week longer in mutant mice than in control mice.

The Memory Enhancement is Reversible

To demonstrate that the enhancement in memory was a direct consequence of CN inhibition, we took advantage of the doxycycline dependence of transgene expression and examined whether its suppression could reverse the memory improvement. Mutant mice displaying enhanced memory with the transgene were withdrawn from doxycycline, then trained and tested again 2 weeks later using a different set of objects. Suppression of CN inhibition in these mutants reversed the increase in exploration of both the displaced [on/off doxycycline versus on doxycycline, F(1,30)=4.495, p<0.05] (FIG. 5F) and the novel object [on/off doxycycline versus on doxycycline, F(1,25)=5.275, p<0.05] (FIG. 5G). Mutant mice not withdrawn from doxycycline and tested again after 2 weeks exhibited enhanced exploration similar to that observed during the first testing session (data not shown). These results indicated that the enhancement in memory is reversible and therefore is a direct consequence of transgene expression in the adult.

Spatial Learning and Memory is Facilitated with the CN Inhibitor

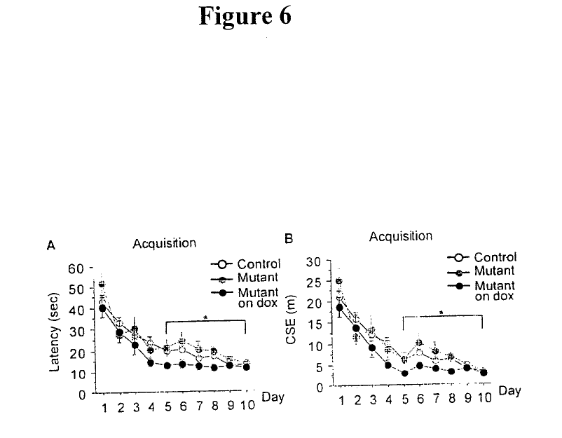

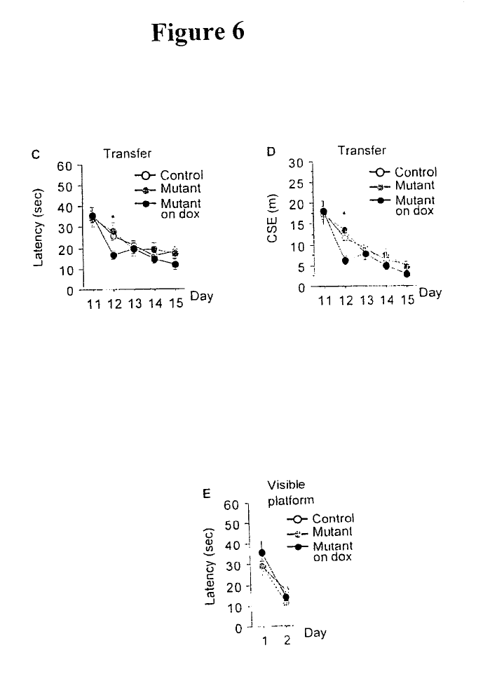

To assess whether memory was enhanced on other behavioral tasks, we used the Morris water maze, a hippocampal-dependent task that challenges spatial learning and memory (Morris et af., 1982). On this task, animals learn the position of a hidden escape platform in a circular pool using distal cues. During acquisition, mice from all groups showed a decrease in escape latency (FIG. 6A) and path length (data not shown) across days, indicating learning of the platform position (all groups, p<0.0001). Similarly after the platform was moved to a different location in the pool (transfer), a decrease in latency and path length was observed across time (FIG. 6C), reflecting learning of the second platform position.

Strikingly however, mutant mice on doxycycline displayed significantly lower escape latencies and shorter path length than control mice (latency, [F(1,70)=4.34, p<0.05]; path length, [F(1,70)=3.98, p<0.05]) or than untreated mutant mice (latency, [F(1,26)=8.97, p<0.01]; path length, [F(1,26)=7.44, p<0.05]). This effect was particularly pronounced between day 5 and day 10 of acquisition (FIG. 6A) and on day 12 of transfer (FIG. 6C). Thus, after 5 days of acquisition, mutant mice on doxycycline showed minimal escape latencies (10-15 s) (no session effect from day 5 to 10; p>0.8), while controls needed almost twice as much training, 4 additional days, to reach comparable levels of performance (significant session effect through day 5 to 10, p<0.05 for all control groups) (FIG. 6A). Similarly, during transfer, mutant mice on doxycycline reached minimal latencies after 2 days (no session effect from day 12 to 15; p>0.09) while controls needed one more day to reach comparable latencies (significant session effect from day 12 to 15; p<0.05 for all control groups) (FIG. 6C). The difference in latency between mutants on doxycycline and controls was seen only on day 5 of acquisition as opposed to day 2 of transfer because, presumably, initial days of acquisition are used for procedural learning that is no longer necessary on transfer (Bannerman et al., 1995). No difference in performance was found between control groups. The analyses of noncognitive parameters also revealed no difference in learning on the cued version of the task (FIG. 6E), in swimming speed or in thigmotaxis (data not shown). These results suggested that the improved performance resulted from enhanced hippocampal-dependent spatial learning and memory and not from changes in motor or motivational/emotional processes.

A decrease in latency or path length, however, does not necessarily indicate place learning as mice can adopt efficient nonspatial strategies such as circular swimming (Gallagher et al., 1993; Wolfer et al., 1998). To specifically evaluate spatial performance, we measured the animal's proximity to the platform with the cumulative search error (CSE), an index of navigation precision. CSE was determined by adding together the distance between the animal and the platform every second for each trial. Consistent with the decrease in latency, all groups showed a decrease in CSE over both training phases (p<0.0001). But again, mutant mice on doxycycline displayed significantly lower CSE than controls [F(1,70)=4.66, p<0.05] or than mutants [F(1,26)=7.49, p<0.05], particularly between acquisition day 5 and 10 (FIG. 6B), and on transfer day 12 (FIG. 6D).

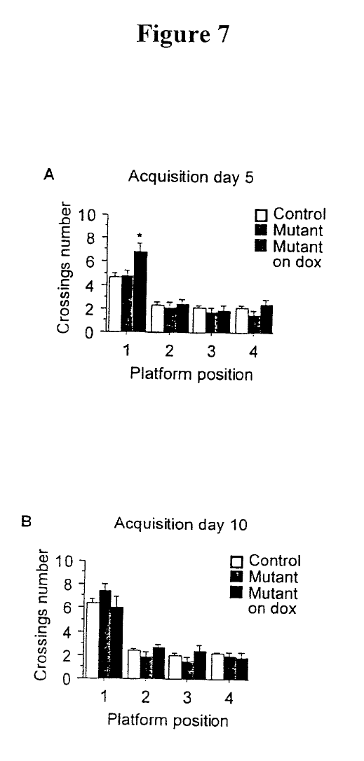

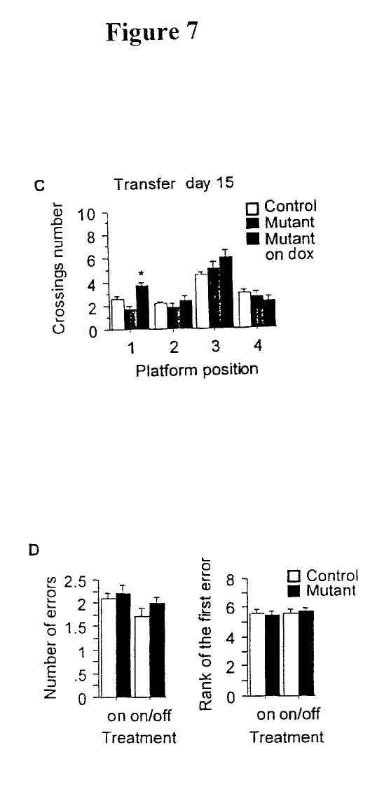

We next evaluated memory for the platform position on days 5, 10, and 15 by removing the platform from the pool and measuring the searching time and number of platform crossings in each quadrant of the pool. For each probe trial, all groups spent more time in the training quadrant than in other quadrants (data not shown), revealing a good memory for the training quadrant. However, on day 5, mutant mice on doxycycline swam across the platform area in the training quadrant significantly more often than controls [F(1,70)=7.52, p<0.01] or than mutants [F(1,26)=7.35, p<0.05], suggesting a more precise memory for the platform position (FIG. 7A). At the end of acquisition (day 10), this difference was no longer apparent as controls and mutants reached levels of performance comparable to mutants on doxycycline (FIG. 7B). Similarly, by the end of transfer (day 15), all groups swam across the platform area in the second training quadrant more often than in other quadrants. Interestingly, mutants on doxycycline also crossed the area where the platform was located during acquisition significantly more often than controls [F(1,70)=6.53, p=0.01] or than mutants [F(1,26)=20.68, p=0.0001], suggesting intact memory for the first platform location (FIG. 7C).

Working Memory is Normal

We next assessed whether memory was improved on very short delays by examining working memory. Working memory is an immediate and rapidly decaying memory thought to be anatomically sustained by a prefrontal cortex-hippocampus network (Olton and Feustle, 1981; Floresco et al., 1997). We used a classical random foraging paradigm for which animals had to retrieve food baits in eight arms of a radial arm maze (Cassel et al., 1998). The optimal strategy for the animals to be rewarded is to remember the arms already visited while performing the task. On this task, no significant difference in the number of errors or the rank of error between groups was found, suggesting no effect of the CN inhibitor on working memory (FIG. 7D).

Discussion

The Inhibition of CN Enhances LTP and Several Phases of Memory

The main finding of our study is that the regulated inhibition of the phosphatase CN leads to enhanced LTP both in vitro and in vivo, and to improved learning and memory storage. The parallel in time course of the increased persistence of LTP in awake animals and of the memory improvement strongly suggest a correlation between the duration of LTP and memory storage. Improved cognitive performance was observed both on spatial and nonspatial hippocampal-dependent tasks, consistent with the multipurpose role of the hippocampus in human declarative memory (Squire, 1992). Moreover, with different tasks, different temporal components of memory were improved. Thus, the complex object recognition task, involving brief training sessions, multiple objects, spatial transfer, and object change, elicited a weak form of memory that was strengthened at early and intermediate time points by the CN inhibitor, but did not persist longer in mutants than in controls. By contrast, a more robust form of memory elicited by a more intense training was maintained and persisted for over a week longer in mutants expressing the CN inhibitor when compared to controls.

Facilitated learning and memory was also observed on the Morris water maze and was evident not only with traditional measurements of spatial performance such as escape latency, but also with more specific aspects of performance such as the precision of navigation. These measures suggested that mutant mice expressing the CN inhibitor retained spatial information more efficiently than controls. The persistent memory for the first platform position associated with the efficient learning of a second platform position suggested an overall enhanced capacity for memory storage with the CN inhibitor. Further, the rapid adaptation to spatial changes observed in mutants expressing the CN inhibitor on both the Moms water maze and the object exploration task suggested increased cognitive flexibility, a process that depends on the hippocampus (Day et al., 1999).

It should be noted that some of our results contrast with those obtained recently in rat where antisense oligonucleotides against CN did not enhance learning and memory on the Morris water maze (Ikegami and Inokuchi, 2000). This discrepancy may be due to different levels of CN inhibition achieved with the two approaches, or to the choice of the animal model or of behavioral paradigms. Finally, on all tasks, the cognitive enhancement observed in the mutant mice was quite remarkable considering the high level of performance in all control groups.

A Molecular Gate for LTP and for Memory Storage

One of the molecular mechanisms allowing transmitted signals to persist or decay is thought to be the balance between phosphatase and kinase activity (Blitzer et al., 1995; Wang and Kelly, 1997). Much evidence suggests that PKA and CN specifically regulate this balance and thereby serve as a gate for LTP (Blitzer et al., 1995,1998; Thomas et al., 1996; Winder et al., 1998). In the current study, we provide further evidence in support of this model by demonstrating that shifting the endogenous balance away from calcineurin activity positively modulates synaptic plasticity in a PKA-dependent manner. Further, together with our previous results (Mansuy et al., 1998a), the current data indicate that altering CN activity transiently in the adult brain is sufficient to positively or negatively control synaptic plasticity and memory storage. The effects observed suggest that CN is essential both for early events of plasticity and memory and for downstream pathways that contribute to persistent changes in plasticity and memory storage.

Mechanistically, early and transient forms of plasticity and memory are known to rely on the covalent modification of pre-existing proteins while long-term forms require activation of transcription factors such as CREB, and protein synthesis (Dash et al., 1990; Bourtchuladze et al., 1994; Impeyetal., 1996). One possible mechanism for the facilitatory effect of the CN inhibitor may be a decrease in the activity of PP1, a protein phosphatase positively regulated by CN through dephosphorylation of inhibitor-1 (I-1). PP1 inhibition has been shown to promote the induction of LTP (Blitzer et al., 1995; 1998), whereas increased PP1 activity, produced by genetic suppression of I-1, has been shown to affect certain forms of LTP in some hippocampal regions (Alien et al., 2000). Since PP1 is effective in modulating CaMKII, a kinase critical for the transmission of postsynaptic signals required for the induction of LTP (Lledoet al., 1995; Otmakhov et al., 1997), it is possible that increased CaMKII activity mediated by lower PP1 activity facilitates the induction of LTP (Makhinson et al., 1999). Raising the signal for the induction of LTP, through genetic upregulation of NMDA-R function, has previously been demonstrated to enhance LTP, learning, and memory (Tang et al., 1999). Our findings suggest that LTP and memory enhancements can be similarly achieved by relieving a constraint downstream of the NMDA-R and that this constraint is exercised by CN.

The effect of the CN inhibitor on long-lasting changes in plasticity and memory may be mediated by modulation of transcriptional control. Thus, the prolonged maintenance of LTP and of memory may arise from augmented CREB transcriptional activity via reduced CREB dephosphorylation by PP1 (Hagiwara et al., 1992). In this context, it is important to note that unlike the phenotypes observed in Drosophila mutants expressing active CREB (Yin et al., 1995), the CN inhibitor did not convert labile memory into long-lasting memory. It was, however, able to strengthen or prolong different phases of memory, suggesting that CN inhibition modulates rather than mediates memory processes.

The PKA and CN pathways may also interact antagonistically at sites other than CREB. For example, CN can inhibit specific isoforms of adenylyl cyclase required for PKA activation (Paterson et al., 1995). Similarly, PKA and CN can regulate, in opposite ways, phosphorylation sites on key proteins in synaptic transmission, such as the NMDA-R (Tong et al., 1995) or the γ-amino-3-hydroxy-5-methyl-4-isoxazole propionate (AMPA) receptor (see for review Yakel, 1997; Banke et al., 2000). The effect of inhibiting CN may also occur through processes additional to or independent of the cAMP pathway. For instance, CN inhibitor may modulate calcium dependent kinases such as CaMKII or PKC through control of intracellular calcium mobilization by regulation of inositol 1,4,5-triphosphate receptors (Cameron et al., 1995). Finally, the PKA/CN gate most likely represents only one of several activator/suppressor mechanisms regulating plasticity and memory (Abel et at., 1998).

A Genetic System for Modulating Cognitive Functions

Several genetic approaches have been used to study the molecular mechanisms of hippocampal functions such as memory (Jerecic et al., 1999). Standard genetic techniques, however, have suffered from the limitation that the genetic modification is permanent. Here we confirmed the usefulness of the rtTA system for such studies by showing that inducible and reversible transgene expression allows temporary improvement of complex cognitive functions and of brain plasticity. The ability to achieve such reversible improvements in the adult animal demonstrates that no permanent changes in neuronal circuits are involved and that the effects result specifically from molecular and biochemical changes elicited by a reduction in CN activity.

### REFERENCES

Abel, T., Nguyen, P. V., Barad, M., Deuel, T. A., Kandel, E. R., and Bourtchouladze, R. (1997). Genetic demonstration of a role for PKA in the late phase of LTP and in hippocampus-based long-term memory. Cell 88, 615-626.

Abel, T., Martin, K. C., Bartsch, D., and Kandel, E. R. (1998). Memory suppressor genes: inhibitory constraints on the storage of long-term memory. Science 279, 338-341.

Allen, P. B., Hvalby, O., Jensen, V., Errington, M. L., Ramsay, M., Chaudhry, F. A., Bliss, T. V., Storm-Mathisen, J., Morris, R. G., Andersen, P., and Greengard, P. (2000). Protein phosphatase-1 regulation in the induction of long-term potentiation: heterogeneous molecular mechanisms. J. Neurosci. 20, 3537-3543.

Banke, T. G., Bowie, D., Lee, H., Huganir, R. L., Schousboe, A., and Traynelis, S. F. (2000). Control of GluR1 AMPA receptor function by cAMP-dependent protein kinase. J. Neurosci. 20, 89-102.

Bannerman, D. M., Good, M. A., Butcher, S. P., Ramsay, M., and Morris, R. G. (1995). Distinct components of spatial learning revealed by prior training and NMDA receptor blockade. Nature 378,182-186.

Bartsch, D., Ghirardi, M,, Skehel, P. A., Karl, K. A., Herder, S. P., Chen, M., Bailey, C. H., and Kandel, E. R. (1995). Aplysia CREB2 represses long-term facilitation: relief of repression converts transient facilitation into long-term functional and structural change. Cell 83, 979-992.

Bienenstock, E. L., Cooper, L. N., and Munro, P. W. (1982). Theory for the development of neuron selectivity: orientation specificity and binocular interaction in visual cortex. J. Neurosci. 2, 32-48.

Blitzer, R. D., Wong, T., Nouranifar, P., Iyengar, P., and Landau, E. M.(1995). Postsynaptic cAMP pathway gates early LTP in hippocampal CA1 region. Neuron 15, 1403-1414.

Blitzer, R. D., Conner, J. H., Brown, G. P., Wong, T., Shenolikar, S., lyengar, R., and Landau, E. M. (1998). Gating of CaMKII by cAMP-regulated protein phosphatase activity during LTP. Science 280, 1940-1942.

Bourtchuladze, R., Frenguelli, B., Blendy, J., Cioffi, D., Schutz, G., and Silva, A. J. (1994). Deficient long-term memory in mice with a targeted mutation of the cAMP-responsive element-binding protein. Cell 79, 59-68.

Buhot, M.-C., and Naili, S. (1995). Changes in exploratory activity following stimulation of hippocampal 5-HT1A and 5-HT1B receptors in the rat. Hippocampus 5,198-208.

Cameron, A. M., Steiner, J. P., Roskams, A. J., Ali, S. M., Ronnett, G. V., and Snyder, S. H. (1995). Calcineurin associated with the inosrtol 1,4,5-trisphosphate receptor-FKBP12 complex modulates Ca!* flux. Cell 83, 463-472.

Cassel, J.-C., Cassel, S., Galani, R., Kelche, C., Will, B., and Jarrard, L. (1998). Fimbria-fornix versus selective hippocampal lesions in rats: effects on locomotor activity and spatial learning and memory. Neurobiol. Learn. Mem. 69, 22-45.

Dash, P. K., Hochner, B., and Kandel, E. R. (1990). Injection of the cAMP-responsive element into the nucleus of Aplysia sensory neurons blocks long-term facilitation. Nature 345, 718-721.

Day, L. B., Weisand, M., Sutherland, R. J., and Schallert, T. (1999). The hippocampus is not necessary for a place response but may be necessary for pliancy. Behav. Neurosci. 113, 914-924.

Errington, M. L., Bliss, T. V. P., Morris, R. J., Laroche, S., and Davis, S. (1997). Long-term potentiation in awake mutant mice. Nature 387, 666-667.

Floresco, S. B., Seamans, J. K., and Phillips, A. G. (1997). Selective roles for hippocampal, prefrontal cortical, and ventral striatal circuits in radial-arm maze tasks with or without a delay. J. Neurosci. 17, 1880-1890.

Gallagher, M., Burwell, R., and Burchinal, M. (1993). Severity of spatial learning impairment in aging: development of a learning index for performance in the Morris water maze. Behav. Neurosci. 107, 618-626.

Gossen, M., Freundlieb, S., Bender, G., Muller, G., Hillen, W., and Bujard, H. (1995). Transcriptional activation by tetracyclines in mammalian cells. Science 268, 1766-1769.

Hagiwara, M., Alberts, A., Brindle, P., Meinkoth, J., Feramisco, J., Deng, T., Karin, M., Shenolikar, S., and Montminy, M. (1992). Transcriptional attenuation following cAMP induction requires PP-1-mediated dephosphorylation of CREB. Cell 70, 105-113.

Huang, Y. Y., and Kandel, E. R. (1994). Recruitment of long-lasting and protein kinase A-dependent long-term potentiation in the CA1 region of hippocampus requires repeated tetanization. Learn. Mem. 1,74-82.

Ikegami, S., and Inokuehi, K. (2000). Antisense DNA against calcineurin facilitates memory in contextual fear conditioning by lowering the threshold for hippocampal long-term potentiation induction. Neuroscience 98, 637-646.

Ikegami, S., Kato, A., Kudo, Y., Kuno, T., Ozawa, F., and Inokuchi, K. (1996). A facilitatory effect on the induction of long-term potentiation in vivo by chronic administration of antisense oligodeoxy-nucleotides against catalytic subunits of calcineurin. Brain Res. Mol. Brain Res. 41,183-191.

Impey, S., Mark, M., Villacres, E. C., Poser, S., Chavkin, C., and Storm, D. R. (1996). Induction of CRE-mediated gene expression by stimuli that generate long-lasting LTP in area CA1 of the hippocampus. Neuron 16, 973-982.

Jerecic, J., Single, F., Kruth, U., Krestel, H., Kolhekar, R., Storck, T., Kask, K., Higuchi, M., Sprengel, R., and Seeburg, P. H. (1999). Studies on conditional gene expression in the brain. Ann. N.Y. Acad. Sci. 868, 27-37.

Kornhauser, J. M., and Greenberg, M. E. (1997). A kinase to remember: dual roles for MAP kinase in long-term memory. Neuron 18, 839-842.

Lee, H. K., Barbarosie, M., Kameyama, K., Bear, M. F., and Huganir, R. L. (2000). Regulation of distinct AMPA receptor phosphorylation sites during bidirectional synaptic plasticity Nature 405, 955-959.

Lisman, J. (1994). The CaM kinase II hypothesis for the storage of synaptic memory. Trends Neurosci. 17, 406-412.

Lledo, P. M., Hjelmstad, G. O., Mukherji, S., Soderling, T. R., Malenka, R. C., and Nicoll, R. A. (1995). Calcium/calmodulin-dependent kinase II and long-term potentiation enhance synaptic transmission by the same mechanism. Proc. Natl. Acad. Sci. USA 92,11175-11179.

Lu, Y. M., Mansuy, I. M., Kandel, E. R., and Roder, J. (2000). Calcineurin mediated long-term depression of GABAergic inhibition underlies the long-lasting increase in the excitability of CA1 hippocampal pyramidal cells induced by LTP. Neuron 26,197-205.

Makhinson, M., Chotiner, J. K., Watson, J. B., and O'Dell, T. J. (1999). Adenylyl cyclase activation modulates activity-dependent changes in synaptic strength and Ca²⁺/calmodulin-dependent kinase II auto-phosphorylation. J. Neurosci. 19, 2500-2510.

Malleret, G., Hen, R., Guillou, J. L, Segu, L, and Buhot, M.-C. (1999). 5-HT1B receptor knock-out mice exhibit increased exploratory activity and enhanced spatial memory performance in the Morris water maze. J. Neurosci. 19, 6157-6168.

Mansuy, I. M., Mayford, M., Jacob, B., Kandel, E. R., and Bach, M. E. (1998a). Restricted and regulated overexpression reveals calcineurin as a key component in the transition from short-term to long-term memory. Cell 92, 39-49.

Mansuy, I. M., Winder, D. G., Moallem, T. M., Osman, M., Mayford, M., Hawkins, R. D., and Kandel, E. R. (1998b). Inducible and reversible gene expression with the rtTA system for the study of memory. Neuron 21, 257-265.

Morris, R. G. M., Garrud, P., Rawlins, J. N. P., and O'Keefe, J. (1982). Place navigation impaired in rats with hippocampal lesions. Nature 297, 681-683.

Mulkey, R. M., Endo, S., Shenolikar, S., and Malenka, R. C. (1994). Involvement of a calcineurin/inhibitor-1 phosphatase cascade in hippocampal long-term depression. Nature 369, 486-488.

Olton, D. S., and Feustle, W. A. (1981). Hippocampal function required for nonspatial working memory. Exp. Brain. Res. 41, 380-389.

Otmakhov, N., Griffith, L., and Lisman, J. E. (1997). Postsynaptic inhibitors of Ca²⁺/calmodulin-dependent protein kinase type II block induction but not maintenance of pairing-induced long-term potentiation. J. Neurosci. 17, 5357-5365.

Papatheodoropoulos, C., and Kostopoulos, G. (2000). Dorsal-ventral differentiation of short-term plasticity in rat CA1 hippocampal region. Neurosci. Lett. 286, 57-60.

Paterson, J. M., Smith, S. M., Harmar, A. J., and Antoni, F. A. (1995). Control of a novel adenylyl cyclase by calcineurin. Biochem. Biophys. Res. Commun. 274, 1000-1008.

Perrino, B. A., Ng, L. Y., and Soderling, T. R. (1995). Calcium regulation of calcineurin phosphatase activity by its B subunit and calmodulin. Role of the autoinhibitory domain. J. Biol. Chem. 270, 340-346.

Soderling, T., and Derkach, V. A. (2000). Postsynaptic protein phosphorylation and LTP. Trends Neurosci. 23, 75-80.

Squire, L. R. (1992). Memory and the hippocampus: a synthesis from findings with rats, monkeys and humans. Psychol. Rev. 99,195-231.

Tang, Y. P., Shimizu, E., Dube, G. R., Rampon, C., Kerchner, G. A., Zhuo, M., Liu, G., and Tsien, J. Z. (1999). Genetic enhancement of learning and memory in mice. Nature 407, 63-69.

Thomas, M. J., Moody, T. D., Makhinson, M., and O'Dell, T. J. (1996). Activity-dependent beta-adrenergic modulation of low frequency stimulation induced LTP in the hippocampal CA1 region. Neuron 17,475-482.

Tong, G., Shepherd, D., and Jahr, C. E. (1995). Synaptic desensitization of NMDA receptors by calcineurin. Science 267,1510-1512.

Vnek, N., and Rothblat, L. A. (1996). The hippocampus and long-term object memory in the rat. J. Neurosci. 16, 2780-2787.

Wang, J. H., and Kelly, P. T. (1997). Postsynaptic calcineurin activity downregulates synaptic transmission by weakening intracellular Ca2+ signaling mechanisms in hippocampal CA1 neurons. J. Neurosci. 17,4600-4611.

Winder, D. G., Mansuy, I. M., Osman, M., Moallem, T. M., and Kandel, E. R. (1998). Genetic and pharmacological evidence for a novel, intermediate phase of long-term potentiation suppressed by calcineurin. Cell 92, 25-37.

Wolfer, D. P., Stagljar-Bozicevic, M., Errington, M. L., and Lipp, H. P. (1998). Spatial memory and learning in transgenic mice: fact or artifact? News Physiol. Sci. 13,118-123.

Yakel, J. L. (1997). Calcineurin regulation of synaptic function: from ion channels to transmitter release and gene transcription. Trends Pharmacol. Sci. 18,124-134.

Yin, J. C., Del Vecchio, M., Zhou, H., and Tully, T. (1995). CREB as a memory modulator: induced expression of a dCREB2 activator isoform enhances long-term memory in Drosophila. Cell 81, 107-115.

Zhuo, M., Zhang, W., Son, H., Mansuy, I., Sobel, R. A., Seidman, J., and Kandel, E. R. (1999). A selective role of calcineurin alpha in synaptic depotentiation in hippocampus. Proc. Natl. Acad. Sci. USA 13, 4650-4655.

What is claimed is:

1. A transgenic nonhuman mammal whose germ or somatic cells contain (i) a first heterologous nucleic acid sequence encoding a transcriptional activator whose expression is under the control of a CaMKIIα promoter and (ii) a second heterologous nucleic acid sequence encoding a protein whose expression is under the control of a promoter responsive to the transcriptional activator in a regulatable manner.

2. The transgenic nonhuman mammal of claim 1, wherein the transcriptional activator comprises rtTA and the promoter of the second nucleic acid sequence comprises a tetracycline-responsive sequence.

3. The transgenic nonhuman mammal of claim 1, wherein the protein encoded by the second nucleic acid sequence is a calcineurin inhibitor.

4. The transgenic nonhuman mammal of claim 3, wherein the calcineurin inhibitor comprises the carboxy-terminal autoinhibitory sequence of calcineurin.

5. The transgenic nonhuman mammal of claim 3, wherein the expression of the calcineurin inhibitor is induced by doxycycline.

6. The transgenic nonhuman mammal of claim 1, wherein the transcriptional activator comprises tTA and the promoter of the second nucleic acid sequence comprises a tetracycline-responsive sequence.

7. The transgenic nonhuman mammal of claim 6, wherein the protein encoded by the second nucleic acid sequence is a calcineurin inhibitor.

8. The transgenic nonhuman mammal of claim 7, wherein the calcineurin inhibitor comprises the carboxy-terminal autoinhibitory sequence of calcineurin.

9. The transgenic nonhuman mammal of claim 7, wherein the expression of the calcineurin inhibitor is repressed by doxycycline.

10. The transgenic nonhuman mammal of claim 1, wherein the mammal is selected from the group consisting of a mouse, a rat, a sheep, a cow, a dog, a pig, and a primate.

11. A mammalian cell comprising (i) a heterologous first nucleic acid sequence encoding a transcriptional activator whose expression is under the control of a CaMKIIα promoter and (ii) a second heterologous nucleic acid sequence encoding a protein whose expression is under the control of a promoter responsive to the transcriptional activator in a regulatable manner.

12. The mammalian cell of claim 11, wherein the transcriptional activator comprises rtTA and the promoter of the second nucleic acid sequence comprises a tetracycline-responsive sequence.

13. The mammalian cell of claim 11, wherein the protein encoded by the second nucleic acid sequence is a calcineurin inhibitor.

14. The mammalian cell of claim 13, wherein the calcineurin inhibitor comprises the carboxy-terminal autoinhibitory sequence of calcineurin.

15. The mammalian cell of claim 13, wherein the expression of the calcineurin inhibitor is induced by doxycycline.

16. The mammalian cell of claim 11, wherein the transcriptional activator comprises tTA and the promoter of the second nucleic acid sequence comprises a tetracycline-responsive sequence.

17. The mammalian cell of claim 16, wherein the protein encoded by the second nucleic acid sequence is a calcineurin inhibitor.

18. The mammalian cell of claim 17, wherein the calcineurin inhibitor comprises the carboxy-terminal autoinhibitory sequence of calcineurin.

19. The mammalian cell of claim 17, wherein the expression of the calcineurin inhibitor is repressed by doxycycline.

20. The mammalian cell of claim 11, wherein the cell is an oocyte, an embryonic stem cell or a neuronal cell.

21. A composition of matter comprising (i) a first nucleic acid encoding a transcriptional activator whose expression is under the control of a CaMKIIα promoter and (ii) a second nucleic acid encoding a protein whose expression is under the control of a promoter responsive to the transcriptional activator in a regulatable manner.

22. The composition of matter of claim 21, wherein the first and second nucleic acids exist in the same nucleic acid molecule.

23. The composition of matter of claim 21, wherein the transcriptional activator comprises rtTA and the promoter of the second nucleic acid sequence comprises a tetracycline-responsive sequence.

24. The composition of matter of claim 21, wherein the protein encoded by the second nucleic acid sequence is a calcineurin inhibitor.

25. The composition of matter of claim 24, wherein the calcineurin inhibitor comprises the carboxy-terminal autoinhibitory sequence of calcineurin.

26. The composition of matter of claim 24, wherein the expression of the calcineurin inhibitor is induced by doxycycline.

27. The composition of matter of claim 21, wherein the transcriptional activator comprises tTA and the promoter of the second nucleic acid sequence comprises a tetracycline-responsive sequence.

28. The composition of matter of claim 27, wherein the protein encoded by the second nucleic acid sequence is a calcineurin inhibitor.

29. The composition of matter of claim 28, wherein the calcineurin inhibitor comprises the carboxy-terminal autoinhibitory sequence of calcineurin.

30. The composition of matter of claim 28, wherein the expression of the calcineurin inhibitor is repressed by doxycycline.

31. A method for determining whether an agent inhibits long-term potentiation in a mammal comprising
(a) administering the agent to the transgenic mammal of claim 5, to which doxycycline has been administered;
(b) measuring the resulting long-term potentiation in the mammal; and
(c) comparing the long-term potentiation so measured to the long-term potentiation measured in a control transgenic mammal of claim 5 to which doxycycline, but no agent, has been administered, a decrease in long-term potentiation relative to the control mammal indicating that the agent inhibits long-term potentiation in a mammal.

32. A method for determining whether an agent inhibits long-term potentiation in a cell comprising
(a) contacting a cultured hippocampal sample from the brain of the transgenic mammal of claim 5 with the agent, wherein the cells of the sample have been exposed to doxycycline;
(b) measuring the resulting long-term potentiation in the hippocampal sample; and
(c) comparing the long-term potentiation so measured to the long-term potentiation measured in a hippocampal sample from a control transgenic mammal of claim 5, wherein the cells of the sample have been exposed to doxycycline, but no agent, a decrease in long-term potentiation relative to the control mammal indicating that the agent inhibits long-term potentiation in a mammal.

33. A method for determining whether an agent inhibits long-term memory formation, retention or recall in a mammal, which comprises
(a) administering the agent to the transgenic mammal of claim 5, to which doxycycline has been administered;
(b) measuring the memory formation, retention, or recall of the mammal in (a) via a behavioral test;
(c) comparing the memory formation, retention, or recall so measured to that of a control transgenic mammal of claim 5 to which doxycycline, but no agent, has been administered, a decrease in memory formation, retention, or recall relative to the control mammal indicating that the agent inhibits memory formation, retention, or recall.

34. The method of claim 33, wherein the behavioral test is selected from the group consisting of the Barnes circular maze, the novel object recognition task, an object exploration task, the Morris water maze and the 8-arm radial maze.
